(19) United States
(12) Patent Application Publication
(10) Pub. No.: US 2012/0308436 A1
(43) Pub. Date: Dec. 6, 2012

(54) ANALYSIS SYSTEM OF BIOLOGICAL PARTICLES IN LIQUID FLOW

(75) Inventor: Masahiko KANDA, Hyogo (JP)

(73) Assignee: BAY BIOSCIENCE KABUSHIKI KAISHA, Kobe-shi (JP)

(21) Appl. No.: 13/486,319

(22) Filed: Jun. 1, 2012

(30) Foreign Application Priority Data
Jun. 3, 2011 (JP) 2011-124845

(51) Int. Cl. G01N 33/48

(52) U.S. Cl. 422/73

(57) ABSTRACT

One embodiment of the present invention is to provide a system for sorting biological particles in a liquid flow running from a flow chamber to a flow cell, which comprises a sheath-liquid tank for storing a mass of a sheath liquid, a fluidic pump being operable to suck the sheath liquid from the sheath-liquid tank, a buffer reservoir being adapted to remove air bubbles from the sheath liquid, and a mass flow controller being operable to supply the sheath liquid free from air bubbles to the flow chamber at a constant flow rate.

### BACKGROUND OF THE INVENTION

The present invention relates to an analysis system of biological particles in a liquid flow running through a flow cell of a flow cytometer or a cell sorter, and in particular, to a feeding system thereof for feeding a sheath liquid and a sample liquid with a flow chamber of the analysis system.

Recent rapid development of biotechnology expands a demand of the flow cytometer and the cell sorter which are more commonly used in the technical fields of medicine and biology for automatic analysis and fractionation of various cell particles or individual cells (referred to simply as “cells” hereinafter). In general, the flow cytometer forms a stream of various cells in line within a sheath flow, which are collected from a living body (blood, etc.) and dyed with a fluorescent labeling reagent, and irradiates one or more laser beams on the stream of the cells to detect information light emitted and/or scattered at the cells (i.e., multi-color fluorescence varied on the fluorescent labeling reagent, forward-scattered light, and side-scattered light) so that each of the cells in the stream is analyzed based upon the information light. The flow cytometer collects and analyzes the information light having identification information of the cells so as to statistically evaluate identification information for a mass of the cells obtained from the sample, thereby allowing diagnosis of a patient's health condition such as a disease of the patient at a cellular level. Furthermore, the cell sorter also uses identification information to selectively charge droplets containing a particular type of the cells to be sorted, and applying a DC electric field across a dropping path of the droplets, thereby selectively retrieving or sorting the desired cells.

In order to collect precise identification information of each cell and apply exactly prompt charge on each droplet, it may be important to align each of the cells in a straight line at a predetermined gap within the liquid flow through the flow cell. Thus, it is demanded to precisely control a given mixture ratio and a flow rate of the sample liquid and the sheath liquid.

However, since a minuscule amount of the sample liquid and the sheath liquid are supplied into the flow cell (the flow rate may be several pico-liter per second), the flow rate is susceptive to temperature and viscosity thereof. Also it is less advantage that the flow rate may be varied by pulsation caused by a pump used for supplying the sample liquid and the sheath liquid. Thus, the constant and stable flow rate of the sample liquid and the sheath liquid controls the running speed of the cells within the sheath flow and the jet flow and to be constant, also adjusts the length of the jet flow JF and a droplet-droplet distance (i.e., a distance between adjacent two droplets D) to be stable, so that a sorting efficiency and a sorting purity of a desired type of cells are improved in the cell sorter.

A conventional liquid feeding system 101 has been proposed as shown in FIG. 5, which includes a sample-liquid feeding mechanism 110 for feeding a sample liquid and a sheath-liquid feeding mechanism 120 for feeding a sheath liquid into the flow chamber in a stable and constant manner without pulsation.

The sample-liquid feeding mechanism 110 includes a sample-liquid plenum chamber 112 for containing the sample liquid in a hermetical manner, an air compressor 114 for supplying pressurized air therein, and a sample pressure sensor 116 for detecting (positive) air pressure within the sample-liquid plenum chamber 112. Upon activation of the air compressor 114, air of positive pressure is applied into the sample-liquid plenum chamber 112 so that the sample liquid therein is pushed by the positive pressure and introduced to a flow chamber 130. During operation, the positive pressure in the sample-liquid plenum chamber 112 is detected by the sample pressure sensor 116, and the sample-liquid feeding mechanism 110 is designed such that the positive pressure is monitored and fedback to the air compressor 114 so that a constant and stable amount of the sample liquid is supplied into the flow chamber 130 without pulsation or fluctuation.

In the meanwhile, for example, as disclosed in Patent Document 1 (JP 2004-77484 A) and illustrated in FIG. 5 herein, the sheath-liquid feeding mechanism 120 includes a sheath-liquid tank 122 for storing a mass of the sheath liquid, a fluidic pump 124 for drawing the sheath liquid from the sheath-liquid tank 122, a pulsation damping chamber 126 for damping the pulsation in the sheath flow from the fluidic pump 124, and a sheath-liquid plenum chamber 128. The fluidic pump 124 typically uses a diaphragm pump so that the sheath liquid drawn by the pump has substantial pulsation. The sheath liquid with substantial pulsation is received in the transiently within the pulsation damping chamber 126 which is hermetically sealed, and subsequently delivered to the sheath-liquid plenum chamber 128 by raising pressure in the pulsation damping chamber 126. The sheath-liquid plenum chamber 128 includes a sheath pressure sensor 125 for detecting (positive) air pressure therein, and a liquid level sensor 127 for detecting the level of the sample liquid. The sheath-liquid plenum chamber 128 is designed to deliver the sheath liquid into the flow chamber 130 at a constant flow rate without pulsation by controlling the pressure and the liquid level in the sheath-liquid plenum chamber 128.

However, since the sheath-liquid feeding mechanism 120 of said Patent Document 1 has such complex structure composed of various components (such as the sheath pressure sensor 125 and the liquid level sensor 127), downsizing and reducing production cost thereof may unlikely be successful. Also when desired to use various types of the sheath liquid, that is, when required to exchange the sheath-liquid plenum chamber 128, complex and time-consuming tasks are necessary to assemble and adjust the feedback mechanism of the sensors. Furthermore, the sheath-liquid feeding mechanism 120 of above-said Patent Document 1 has another problem that when designed to control the constant flow rate of the sheath liquid by adjusting the pressure within the sheath-liquid plenum chamber 128, the flow rate thereof is often influenced by viscosity which varies with temperature of the sheath liquid.

One embodiment of the present invention is to address the above-mentioned drawbacks, and to provide the sample-liquid feeding system and the sheath-liquid feeding system for feeding the sample liquid and the sheath liquid with the flow chamber, respectively, at a constant flow rate without pulsation, which are structured in a convenient manner. Also, another embodiment of the present invention is to realize the sample-liquid feeding system and the sheath-liquid feeding system, which can exchange the sample liquid and sheath liquid with different ones in a simplified manner.

### SUMMARY OF THE INVENTION

One aspect of the present invention is to provide a system for sorting biological particles in a liquid flow running from a flow chamber to a flow cell, which comprises a sheath-liquid tank for storing a mass of a sheath liquid, a fluidic pump being operable to suck the sheath liquid from the sheath-liquid tank, a buffer reservoir being operable to remove air bubbles from the sheath liquid, and a mass flow controller being operable to supply the sheath liquid free from air bubbles to the flow chamber at a constant flow rate.

Preferably, the buffer reservoir is adapted to remove air bubbles from the sheath liquid, and keep the sheath liquid at a constant temperature. More preferably, the system further may include a liquid-pressure adjusting valve. The mass flow controller may preferably be provided adjacent the flow chamber.

Another aspect of the present invention is to provide a system for analyzing biological particles in a liquid flow running from a flow chamber to a flow cell, which may comprise an air compressor operable to compress ambient air, an air regulator operable to regulate pressure of the compressed air at a constant level, a positive-pressure chamber hermetically sealed and filled up with the air regulated at the constant level, a sample-liquid reservoir detachably provided within the positive-pressure chamber, for receiving a sample liquid, and a sample-liquid conduit extending from the sample-liquid reservoir to the flow chamber. The sample-liquid reservoir may receive thermally controlled liquid around the sample-liquid reservoir, which is operable to keep the sample liquid at a constant temperature.

Preferably, the sample-liquid reservoir may be arranged right over the flow chamber. More preferably, the flow chamber may include a sample guiding conduit hermetically sealed therewith. Also, a three-way may be provided between the sample-liquid reservoir and the sample guiding conduit. Furthermore, the system may be adapted to reversely draw the sheath liquid from the flow chamber through the sample guiding conduit so as to remove cell particles adhered on the sample guiding conduit. In addition the sample-liquid reservoir may have a bottom portion of which inner diameter is reduced towards a downward direction. Also, the sample-liquid reservoir may have a bottom member of resilient material, and the sample guiding conduit may have a disposable sharp metal needle, which is pierced into the bottom member of the sample-liquid reservoir allowing liquid communication between the sample-liquid reservoir and the flow chamber.

### BRIEF DESCRIPTION OF THE DRAWINGS

FIG. 1 is a schematic overview of a first embodiment of a cell sorter according to the present invention.

FIGS. 2A-2C are enlarged cross sectional views of various sample guiding conduits.

FIG. 3 is a partial schematic view of the cell sorter illustrating a sample-liquid reservoir, a positive-pressure chamber, and a flow chamber.

FIG. 4 is a partial schematic view of the sample-liquid reservoir and an upstream sample conduit of Modification.

FIG. 5 is a schematic overview of a conventional cell sorter.

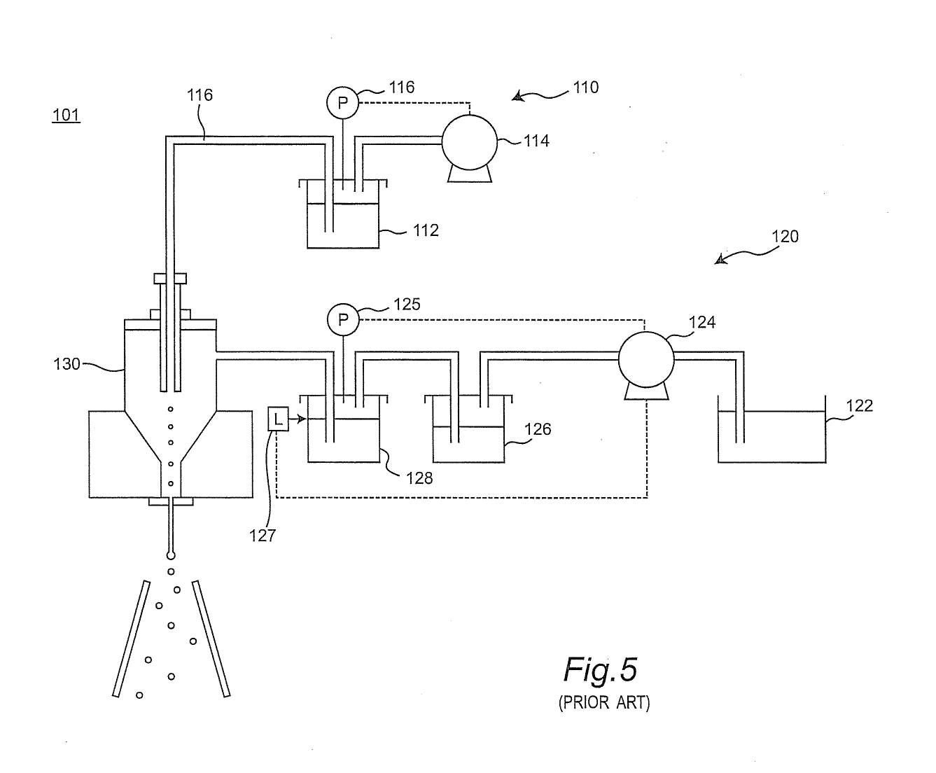

### BRIEF DESCRIPTION OF REFERENCE NUMERALS

1: cell sorter, 10: sample-liquid feeding mechanism, 11: air compressor, 12 buffer tank, 13: air regulator, 14: positive-pressure chamber, 15: sample-liquid reservoir, 16: sample-liquid conduit, 17 sample-liquid clump, 18: thermal controller, 19: agitator, 20: sheath-liquid feeding mechanism, 21: sheath-liquid tank, 22: fluidic pump, 23: liquid-pressure adjusting valve, 24: buffer reservoir, 25: mass flow controller, 30: fluid flow mechanism, 31: flow cell, 32: piezoelectric device, 33: discharging electrode, 35: flow chamber, 36: sample guiding conduit, 37: constriction, 38: three-way valve, 40: sorting mechanism, 42: deflectors, JF: jet flow, D: droplet

### DETAILED DESCRIPTION OF THE EMBODIMENTS

Referring to attached drawings, embodiments of an analysis system of biological particles in liquid glow according to the present invention will be described herein. Although an example of a cell sorter will be described hereinafter for facilitating understandings of the present invention, it may be applied equally to a flow cytometer. In the description, a couple of terms for indicating the directions (for example, “upper” or “lower”) are conveniently used just for clear understandings, it should not be interpreted that those terms limit the scope of the present invention.

### Embodiment 1

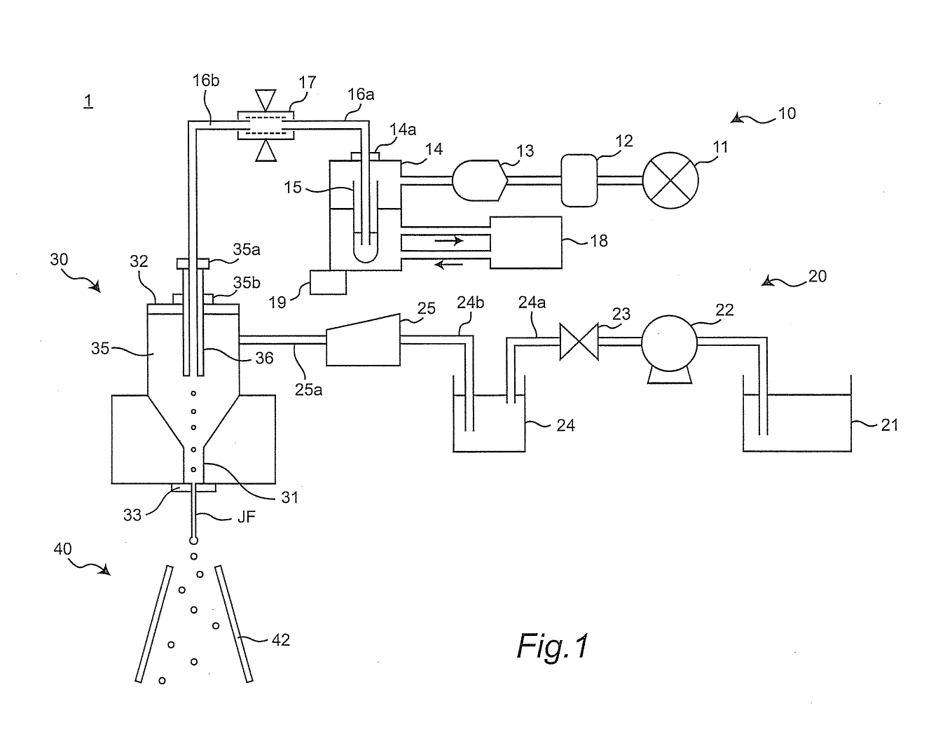

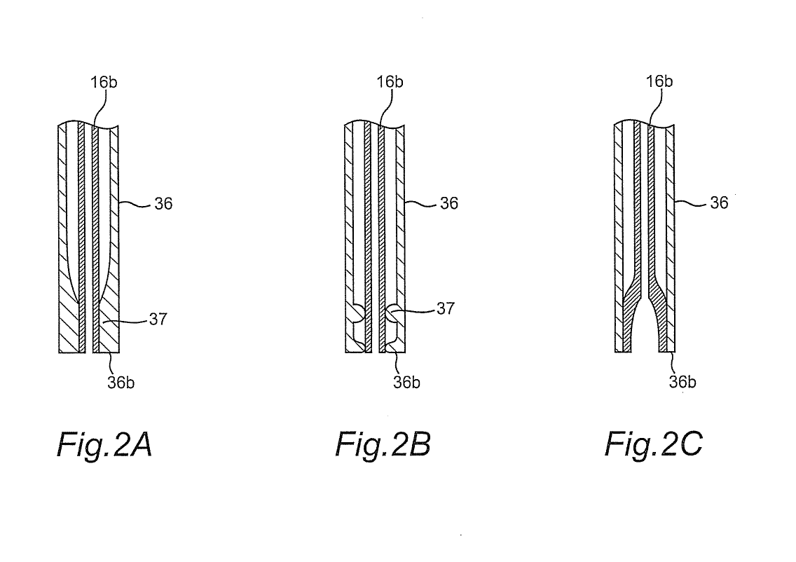

With reference to FIGS. 1 and 2, a first embodiment of the cell sorter according to the present invention will be described herein in detail. FIG. 1 is a schematic overview of the cell sorter 1. The cell sorter 1 includes, in general, a sample-liquid feeding mechanism 10, a sheath-liquid feeding mechanism 20, a fluid flow mechanism 30, an optical mechanism (not shown), and a sorting mechanism 40.

The fluid flow mechanism 30 is adapted to form a sheath flow surrounding a sample flow containing and aligning various cells dyed with a fluorescent labeling reagent in line, and to form a series of droplets D (each containing the cell) from a tip of a jet flow JF, by oscillation of the piezoelectric device 32.

The optical mechanism is structured to irradiate a plurality of laser beams having wavelengths different from one another onto each of the cells aligned in line within the flow cell 31, and to receive a scattering beam and a fluorescent beam which are unique to each of the particular cells.

The sorting mechanism 40 is adapted to analyze the informative signals unique to each of the cells, apply an electronic charge thereto at a predetermined time interval through a discharging electrode 33, and sort a particular kind of cells by providing a given voltage with a pair of deflectors 42 therebetween.

Since any types of the above optical mechanism and the sorting mechanism 40 have been proposed so far and may be used with the present invention, no further description will be made therefor, and the sample-liquid feeding mechanism 10 and the sheath-liquid feeding mechanism 20 will mainly be explained in detail hereinafter.

<Sample-Liquid Feeding Mechanism> The sample-liquid feeding mechanism 10 according to the present invention includes an air compressor 11 for compressing air, a buffer tank 12 for receiving compressed air to remove pulsation therein, an air regulator 13 for regulating pressure of the compressed air at a constant level (about 10 psi-100 psi), and a positive-pressure chamber 14 hermetically sealed and filled up with air of regulated constant pressure.

Provided within the positive-pressure chamber 14 is a sample-liquid reservoir 15 for receiving the sample liquid under the pressurized atmosphere. Since the sample-liquid reservoir 15 is open in the positive-pressure chamber 14 without being hermetically sealed, positive pressure in the chamber 14 pushes directly onto the sample liquid in the sample-liquid reservoir 15, which, in turn, is delivered to a flow chamber 35 through upstream and downstream sample-liquid conduits 16a, 16b and a sample-liquid clump 17. The compressed air has pulsation removed in the buffer tank 12 and pressure regulated at a constant level within the air regulator 13, so that the sample liquid can be delivered into the flow chamber 35 with removed pulsation at an even flow rate in a controlled manner.

The sample-liquid reservoir 15 is adapted such that it is detachably replaced in the positive-pressure chamber 14 with another one quite easily. That is, if desired to exchange one sample liquid to another, firstly the sample-liquid clump 17 is closed, then the original sample-liquid reservoir 15 is taken out of the positive-pressure chamber 14, and lastly another new sample-liquid reservoir 15 is replaced into the positive-pressure chamber 14. This substantially simplifies the task for exchanging the sample liquid. Yet, since the sample liquid remained in the upstream and downstream sample-liquid conduits 16a, 16b and the sample-liquid clump 17, those components should be replaced with new ones or irrigated/cleaned.

Received within the positive-pressure chamber 14 is fluid having substantial specific heat capacity such as water (thermally controlled water) so as to surround the sample-liquid reservoir 15. Also, the positive-pressure chamber 14 is designed such that thermally controlled water is circulated between the sample-liquid reservoir 15 and a thermal controller 18 provided outside thereof. This allows the cells in the sample liquid to be maintained at a predetermined range of temperature (e.g., about 4-42 degrees C.), and to be kept in a viable condition of the cells, which in turn, improves accuracy for sorting cells by mean of the sorting mechanism 40.

The positive-pressure chamber 14 is preferably provided with an agitator 19 for agitating the sample liquid within the sample-liquid reservoir 15, thereby keeping it at uniform temperature and preventing agglomeration of the cells therein.

Also, the positive-pressure chamber 14 is preferably provided with a first sealing connector 14a surrounding the upstream sample-liquid conduit 16a for securing hermetical or liquid-tight seal between the positive-pressure chamber 14 and the upstream sample-liquid conduit 16a. Similarly, the flow chamber 35 is preferably provided with a second sealing connector 35a for hermetically or liquid-tightly sealed connection between the downstream sample-liquid conduit 16b and a sample guiding conduit 36. Also, the flow chamber 35 is provided with a third sealing connector 35b for hermetically or liquid-tightly sealed connection between the sample guiding conduit 36 and the flow chamber 35.

The upstream and downstream sample-liquid conduits 16a, 16b may be made from flexible tubes of material such as PEEK resin, TEFLON®, silicone rubber, TYGON®, having an inner diameter of about 200 μm through 400 μm. On the other hand, the sample guiding conduit 36 may be made of relatively hard material such as metal and glass.

According to the present embodiment of the invention, the sample guiding conduit 36 is precisely aligned to the center of the flow cell 31, and the downstream sample-liquid conduit 16b is adapted to detachably inserted into the sample guiding conduit 36. Thus, upon exchanging the sample-liquid reservoir 15, and if it is required to exchange also the upstream and downstream sample-liquid conduits 16a, 16b, the downstream sample-liquid conduit 16b can be inserted into and guided by the sample guiding conduit 36 into a precisely aligned at a center position of the flow cell 31. This allows easy exchange of the sample liquid and eliminates a complicated task for aligning the downstream sample-liquid conduit 16b with the flow cell 31, in comparison to the conventional approach that the old sample guiding conduit itself is exchanged and the new sample guiding conduit is again aligned at the center of the flow chamber 35.

As above, in order to exchange the sample liquid, it is needed to exchange the sample-liquid reservoir 15, the upstream and downstream sample-liquid conduits 16a, 16b, and the sample-liquid clump 17 with another ones to eliminate possible contamination of the old sample liquid being remained or carried over within the upstream and downstream sample-liquid conduits 16a, 16b, thereby removing adverse impact on detection and/or sorting of the new cells after exchanging the sample-liquid.

As shown in FIGS. 2A and 2B, the sample guiding conduit 36 may have a constriction 37 adjacent a bottom thereof to facilitate precise alignment of the downstream sample-liquid conduit 16b on the center of the flow cell 31 when inserting the downstream sample-liquid conduit 16b into the sample guiding conduit 36. Alternatively as shown in FIG. 2C, the downstream sample-liquid conduit 16b may have an enlarged tip having extended diameter or greater diameter downwardly so that each of the particulate cells has an even or constant falling/running speed that is irrelevant to a radial position where the cell comes out of the tip of sample guiding conduit 36.

<Sheath-Liquid Feeding Mechanism> The sheath-liquid feeding mechanism 20 according to the present embodiment of the invention generally includes, as illustrated in FIG. 1, a sheath-liquid tank 21 for storing a mass of the sheath liquid, a fluidic pump 22 in fluidic communication with the sheath-liquid tank 21, a liquid-pressure adjusting valve 23, and a buffer reservoir 24 for removing air bubbles from the sheath liquid, and a liquid mass flow controller 25 for supplying the sheath liquid free from air bubbles to the flow chamber 35 at an even or constant flow rate.

The fluidic pump 22 may be any type of pumps as long as sucking the sheath liquid from the sheath-liquid tank 21 such as a diaphragm pump. The liquid-pressure adjusting valve 23 is provided for dumping pulsation with the sheath liquid sucked from the sheath-liquid tank 21, and may be, for example, an orifice in a conduit extending between the fluidic pump 22 and the buffer reservoir 24. The buffer reservoir 24 is adapted for remove air bubbles from the sheath liquid, and the first conduit 24a coming into the buffer reservoir 24 has a bottom portion which is located at a higher level of a bottom portion of the second conduit 24b coming out of the buffer reservoir 24, and both of the bottom portions are below the surface of the sheath liquid in the buffer reservoir 24.

The mass flow controller 25 includes a capillary and a pair of resistive heating elements wound at upstream and downstream portions of the capillary. The mass flow controller 25 is adapted to apply a voltage across each of the resistive heating elements, detect thermal difference between the heating elements based upon the current therethrough, determine the flow rate through the capillary based upon thermal difference, and then feedback an actuator valve in fluid communication with the capillary to control the actuator valve whether to be opened or closed based upon the flow rate. This allows the sheath flow to be supplied into the flow chamber 35 at an even or constant flow rate. Such mass flow controllers may be used which are available through Horiba Ltd. Japan, as LV-F series of a mass flow controller.

According to the sheath liquid feeding mechanism 120 of the aforementioned Patent Document 1, in order to supply the sheath liquid at an even or constant flow rate, it is required to provide the sheath pressure sensor 125 for detecting positive air pressure in the sheath-liquid plenum chamber 128 and as well as the liquid level sensor 127 for detecting the level of the sample liquid, which avoids the structure of the sheath liquid feeding mechanism being complicated and downsized. On the other hand, according to one embodiment of the present invention, a mass flow of the sheath liquid can be precisely controlled by means of the mass flow controller 25 which has the simplified and downsized structure. Also, according to the sheath liquid feeding mechanism 120 of the aforementioned Patent Document 1, when required to exchange the sheath liquid, a rather complex and time-consuming task is needed to rearranging the sheath pressure sensor 125 and the liquid level sensor 127 on the sheath-liquid plenum chamber 128 so that the fluidic pump 124 is appropriately fedback with information or signals from the sheath pressure sensor 125 and the liquid level sensor 127. However, according to the present embodiment, the mass flow of the sheath flow is directly measured by the mass flow controller 25, while eliminating the complex and time-consuming tasks as Patent Document 1. Furthermore, the pulsation in the sheath liquid supplied to the flow chamber 35 can advantageously be minimized or fully removed by using the mass flow controller 25. Also, as clearly understood from the operation principle of the mass flow controller 25, even if viscosity of the sheath liquid varies along with temperature thereof, the mass flow thereof receives no impact, and therefore, the sheath liquid can be supplied to the flow chamber 35 in a stable and constant manner.

In addition, a thermal controller (not illustrated) may be provided around the buffer reservoir 24 (which is provided for removing air bubbles from the sheath liquid), for keeping the sheath liquid at a predetermined range of temperature (e.g., about 4-42 degrees C.). Also, temperature of the sheath liquid and the sample liquid may be controlled as being substantially the same. As discussed above, temperature of the sheath liquid gives substantial impact on the running speed of the cell (or the flow rate of the sheath flow), the length of the jet flow, and the distance between adjacent droplets D. Thus, in the cell sorter so structured, by keeping the temperature of the sheath liquid at an even level, the sort and recovery rate of the sorted cells are fairly improved and the purity thereof are substantially stabilized.

In order to avoid temperature of the sheath liquid varying closer to a room temperature while running from the thermal controller provided around the buffer reservoir 24 to the flow chamber 35, it is advantageous to design the conduit 24b from the buffer reservoir 24 to the mass flow controller 25 as well as the conduit 25a from the mass flow controller 25 to the flow chamber 35 to be minimized. More preferably, the buffer reservoir 24 and the mass flow controller 25 are arranged adjacent to the flow chamber 35, and even more preferably, the buffer reservoir 24 and the mass flow controller 25 are received in the same space (not shown) as one in which the flow chamber 35 is housed. The cell sorter so structured controls the temperature of the sheath liquid at substantially the same level so that the sort efficiency and recovery rate are increased and the purity of the sorted cells is substantially stabilized.

### Embodiment 2

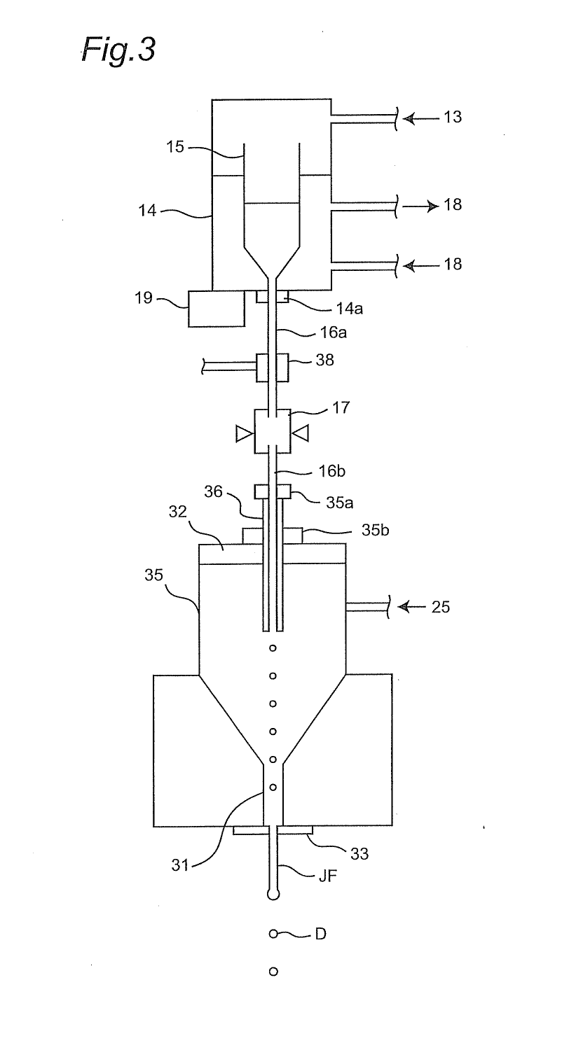

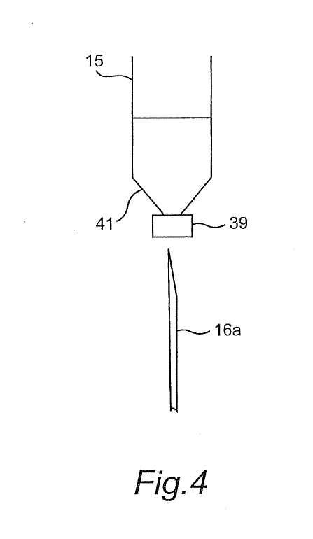

With reference to FIGS. 3 and 4, a second embodiment of the cell sorter according to the present invention will be described herein in detail. The cell sorter 1 of the second embodiment is similar to one of the first embodiment except that the positive-pressure chamber 14 receiving the sample-liquid reservoir 15 is arranged right above the flow chamber 35. Therefore, the duplicated description in detail for the common features will be eliminated. Similar components are denoted with similar reference numerals throughout the description.

FIG. 3 is a partial schematic view of the cell sorter 1 illustrating the sample-liquid reservoir 15 within the positive-pressure chamber 14 and the flow chamber 35. As shown, the positive-pressure chamber 14 is positioned immediately above the flow chamber 35 and the upstream sample-liquid conduit 16a extends downwardly from the bottom of the flow chamber 35.

Air in the positive-pressure chamber 14 is compressed at a constant pressure by the air regulator 13 and the sample liquid in the sample-liquid reservoir 15 is delivered directly to the flow chamber 35 due to action of the gravity and the positive pressure within the chamber 14. Similar to the first embodiment, the positive-pressure chamber 14 is structured such that thermally controlled water therein is circulated from and to the thermal controller 18, thereby keeping the sample-liquid reservoir 15 at the constant temperature. Also the positive-pressure chamber 14 may be provided with an agitator 19 for mixing the sample liquid. Thus, the positive-pressure chamber 14 is preferably designed to keep the sample liquid at the even temperature and improve the surviving rate of the cell particles therein.

Optionally, the sample-liquid conduit 16 may be structured to have a three-way valve 38 in between, allowing the sheath liquid to clean the sample-liquid conduit 16 by passing the sheath liquid therethrough. Also, as shown in FIG. 4, the sample-liquid reservoir 15 may have a bottom member 39 (a rubber plug) of resilient material, which can be inserted or pierced by a disposable sharp metal needle on the upstream sample-liquid conduit 16a. This facilitates assembly of the upstream sample-liquid conduit 16a with the sample-liquid reservoir 15. Also the sample-liquid reservoir 15 may be funneled having a bottom portion of which inner diameter is reduced towards a downward direction. This helps finishing up the sample liquid without remaining in the sample-liquid reservoir 15.

As discussed above, the sample-liquid reservoir 15 can easily be exchanged with another one and the sample-liquid conduit 16 may be cleaned with the sheath liquid. The three-way valve 38 has three ports, including first one connected to the upstream sample-liquid conduit 16a (the sample-liquid reservoir 15), second one connected to the downstream sample-liquid conduit 16b (the sample guiding conduit 36), and third one connected to a reversing pump (not shown). When desired to exchange the sample liquid with another one and to clean the sample-liquid conduit 16, the reversing pump is driven to draw the sheath liquid from the flow chamber 35 through the downstream sample-liquid conduit 16b and the clump 17 in a reverse direction so as to flush the cells on the sample-liquid conduit and the clump 17 in a simple manner, thereby avoiding so-called “carry-over” of the cells.

According to the second embodiment of the present invention, since the sample-liquid reservoir 15 is arranged right over the flow chamber 35, the length of the upstream and downstream sample-liquid conduits 16a, 16b can be reduced so as to minimize thermal fluctuation of the sample liquid influenced by the room temperature during running through the upstream and downstream sample-liquid conduits 16a, 16b, thereby keeping the sample liquid at an even or constant level. Similarly, the buffer reservoir 24 and the mass flow controller 25 may preferably be arranged also adjacent the flow chamber 35 for minimizing thermal fluctuation of the sheath liquid as well. As discussed above, the sample liquid and the sheath liquid receives no thermal influence from the room temperature, and are introduced into the flow chamber 35 at an even temperature, which improves the recovery rate of the sorted cells and substantially stabilizes the purity thereof.

What is claimed is:

1. A system for sorting biological particles in a liquid flow running from a flow chamber to a flow cell, said system comprising:
a sheath-liquid tank for storing a mass of a sheath liquid;
a fluidic pump being operable to suck the sheath liquid from said sheath-liquid tank;
a buffer reservoir being operable to remove air bubbles from the sheath liquid; and
a mass flow controller being operable to supply the sheath liquid free from air bubbles to said flow chamber at a constant flow rate.

2. The system according to claim 1, wherein said buffer reservoir is adapted to remove air bubbles from the sheath liquid, and keep the sheath liquid at a constant temperature.

3. The system according to claim 1, further comprising a liquid-pressure adjusting valve being operable to dump pulsation in the sheath liquid from said fluidic pump.

4. The system according to claim 1, wherein said mass flow controller is provided adjacent said flow chamber.

5. A system for analyzing biological particles in a liquid flow running from a flow chamber to a flow cell, said system comprising:
an air compressor operable to compress ambient air;
an air regulator operable to regulate pressure of the compressed air at a constant level;
a positive-pressure chamber hermetically sealed and filled up with the air regulated at the constant level;
a sample-liquid reservoir detachably provided within said positive-pressure chamber, for receiving a sample liquid; and
a sample-liquid conduit extending from said sample-liquid reservoir to said flow chamber,
wherein said sample-liquid reservoir receives thermally controlled liquid around said sample-liquid reservoir, which is operable to keep the sample liquid at a constant temperature.

6. The system according to claim 5, wherein said sample-liquid reservoir is arranged right over said flow chamber.

7. The system according to claim 5, where said flow chamber includes a sample guiding conduit hermetically sealed therewith, wherein a three-way is provided between said sample-liquid reservoir and said sample guiding conduit, and wherein the system is adapted to reversely draw the sheath liquid from said flow chamber through said sample guiding conduit so as to remove cell particles adhered on said sample guiding conduit.

8. The system according to claim 5, wherein said sample-liquid reservoir has a bottom portion of which inner diameter is reduced towards a downward direction.

9. The system according to claim 5, wherein said sample-liquid reservoir has a bottom member of resilient material, and wherein said sample guiding conduit has a disposable sharp metal needle, which is pierced into the bottom member of said sample-liquid reservoir for allowing liquid communication between said sample-liquid reservoir and said flow chamber.
